(19) United States
(12) Patent Application Publication
(10) Pub. No.: US 2025/0068896 A1
(43) Pub. Date: Feb. 27, 2025

(54) METHOD AND APPARATUS FOR PREDICTING TARGET TASK BASED ON MOLECULAR DESCRIPTOR, AND METHOD OF TRAINING PREDICTION MODEL FOR PREDICTING TARGET TASK

(71) Applicants: SAMSUNG ELECTRONICS CO., LTD., Suwon-si (KR); RESEARCH & BUSINESS FOUNDATION SUNGKYUNKWAN UNIVERSITY, Suwon-si (KR)

(72) Inventors: Youngchun KWON, Suwon-si (KR); Seokho KANG, Gwacheon-si (KR); Jin Woo KIM, Suwon-si (KR); Seung Min BAEK, Suwon-si (KR); Joonhyuk CHOI, Suwon-si (KR); Taesin HA, Suwon-si (KR)

(73) Assignees: SAMSUNG ELECTRONICS CO., LTD., Suwon-si (KR); RESEARCH & BUSINESS FOUNDATION SUNGKYUNKWAN UNIVERSITY, Suwon-si (KR)

(21) Appl. No.: 18/811,181

(22) Filed: Aug. 21, 2024

(30) Foreign Application Priority Data
Aug. 21, 2023 (KR) 10-2023-0109133

(51) Int. Cl. G06N 3/0499; G06N 3/045; G06N 3/0895; G16C 20/10; G16C 20/70

(52) U.S. Cl. CPC G06N 3/0499; G06N 3/045; G06N 3/0895; G16C 20/10; G16C 20/70

(57) ABSTRACT

Provided is a method of training a prediction model, the method including obtaining molecular descriptors of molecules based on a molecular database, pre-training a pre-training neural network based on the molecular descriptors, and adjusting the pre-training neural network such that the pre-training neural network matches a target task, by applying a training data set labeled corresponding to the target task to the pre-trained pre-training neural network.

### CROSS-REFERENCE TO RELATED APPLICATION

This application claims priority to Korean Patent Application No. 10-2023-0109133, filed on Aug. 21, 2023, in the Korean Intellectual Property Office, the entire disclosure of which is incorporated herein by reference for all purposes.

### BACKGROUND

### 1. Field

Example embodiments of the present disclosure relate to predicting a target task based on a molecular descriptor, and training a prediction model for predicting the target task.

### 2. Description of Related Art

A neural network may refer to a computing architecture that models a biological brain. As neural network advances, electronic devices used in various fields may use a neural network-based model to analyze input data and extract and/or output valid information.

For example, predicting the physical properties and/or yield of a material may involve numerous experiments performed by a great number of human resources, however, the accuracy of the physical properties predicted based on the results of these experiments may not be very high. In addition, when the physical properties and/or yield are predicted using a pre-training model trained using unlabeled molecular structures, the physical properties or yield may significantly change even with a small change in a material structure, and accordingly, it is difficult for a training model to have a high accuracy through training.

### SUMMARY

One or more embodiments may address at least the above problems and/or disadvantages and other disadvantages not described above. Also, the embodiments are not required to overcome the disadvantages described above, and an embodiment may not overcome any of the problems described above.

According to an aspect of an example embodiment, there is provided a method of training a prediction model, the method including obtaining molecular descriptors of molecules based on a molecular database, pre-training a pre-training neural network based on the molecular descriptors, and adjusting the pre-training neural network such that the pre-training neural network matches a target task, by applying a training data set labeled corresponding to the target task to the pre-trained pre-training neural network.

The pre-training of the pre-training neural network includes reducing a dimensionality of the molecular descriptors based on a principal component analysis (PCA), and pre-training the pre-training neural network based on the molecular descriptors with the reduced dimensionality as pseudo labels of a molecular graph.

The reducing of the dimensionality of the molecular descriptors may include generating a pre-training data set including molecular graphs respectively corresponding to the molecules and first latent vectors corresponding to the molecular graphs, by reducing the dimensionality of the molecular descriptors based on the PCA.

The pre-training of the pre-training neural network may include assigning the first latent vectors to pseudo labels of the molecular graphs respectively corresponding to the molecules, and pre-training the pre-training neural network to predict a target pseudo label corresponding to the target molecule, based on the pseudo labels.

The pre-training of the pre-training neural network may include inputting input information corresponding to structural information of a target molecule to the pre-training neural network and outputting a molecular representation vector corresponding to the target molecule, predicting a second latent vector corresponding to a target pseudo label of the input information by applying the molecular representation vector to a linear head, and training at least one of the pre-training neural network or the linear head based on a difference between the first latent vectors and the second latent vector.

The training of at least one of the pre-training neural network or the linear head may include training at least one of the pre-training neural network or the linear head based on an objective function based on a weighted mean squared error (WMSE) between the first latent vectors and the second latent vector.

The training data set labeled corresponding to the target task may include a training data set labeled with a target chemical reaction corresponding to the target task and a target yield corresponding to the target chemical reaction.

The pre-training neural network may include at least one of a graph neural network (GNN) or a large language model (LLM).

The target task may include at least one of a prediction of a yield of a target chemical reaction corresponding to the target task, a prediction of a reaction condition of the target chemical reaction, or a prediction of physical properties of the target chemical reaction.

According to another aspect of an example embodiment, there is provided a method of predicting a target task, the method including receiving a query chemical reaction corresponding to a set of a reactant and a product, and predicting a target task corresponding to the query chemical reaction by inputting the query chemical reaction to a prediction model including at least one pre-training neural network that is pre-trained, wherein the prediction model is adjusted to predict a result corresponding to the target task by applying a training data set labeled corresponding to the target task to the pre-training neural network.

The prediction model may be configured to predict a yield corresponding to the query chemical reaction based on the query chemical reaction corresponding to the set of the reactant and the product being input.

The reactant may include molecular graphs corresponding to a plurality of reactant molecules corresponding to different reactions, and the product may include a single molecular graph corresponding to a product molecule.

The molecular graphs and the single molecular graph respectively may include node vectors corresponding to node features corresponding to heavy atoms in a molecule, and edge vectors corresponding to edge features corresponding to chemical bonds between the heavy atoms in the molecule.

The node features may include at least one of an atom type of the heavy atoms, formal charges of the heavy atoms, a degree of the heavy atoms, a hybridization of the heavy atoms, a number of atoms adjacent to the heavy atoms, a valence of the heavy atoms, a chirality of the heavy atoms, associated ring sizes of the heavy atoms, whether the heavy atoms donate or accept electrons, whether the heavy atoms are aromatic, or whether the heavy atoms include a ring.

The edge features may include at least one of a bond type of the chemical bonds between the heavy atoms, a stereochemistry of the chemical bonds between the heavy atoms, whether a ring is in the chemical bonds between the heavy atoms, or whether the chemical bonds between the heavy atoms are conjugated.

The prediction model may include the at least one pre-training neural network configured to output molecular query representation vectors by processing a query molecular graph within the query chemical reaction, at least one fully-connected layer respectively corresponding to the at least one pre-training neural network and configured to output high-dimensional molecular representation vectors corresponding to the molecular query representation vectors, and a feedforward neural network (FNN) configured to integrate the high-dimensional molecular representation vectors and output a prediction result corresponding to the target task by a representation vector of a chemical reaction obtained from the integrated high-dimensional molecular representation vectors.

The prediction model may further include a one-hot-encoding layer corresponding to each of a temperature condition, a pressure condition, and a solvent condition that correspond to the query chemical reaction, and the one-hot-encoding layer may be between the at least one fully-connected layer and the FNN.

The training data set labeled corresponding to the target task may include a training data set labeled with a target chemical reaction corresponding to the target task and a target yield corresponding to the target chemical reaction.

According to another aspect of an example embodiment, there is provided a non-transitory computer-readable storage medium storing instructions that, when executed by a processor, cause the processor to perform the method.

According to another aspect of an example embodiment, there is provided an apparatus for predicting a yield, the apparatus including a communication interface configured to receive a query chemical reaction corresponding to a set of a reactant and a product, and a processor configured to predict a yield corresponding to the query chemical reaction by inputting the query chemical reaction to a prediction model including a pre-trained graph neural network (GNN), wherein the prediction model is adjusted to predict the yield corresponding to the query chemical reaction by applying a training data set labeled corresponding to the predicted yield to the GNN.

### BRIEF DESCRIPTION OF THE DRAWINGS

The above and/or other aspects will be more apparent by describing certain embodiments with reference to the accompanying drawings, in which:

FIG. 1 is a flowchart illustrating a method of training a prediction model for performing a target task according to an example embodiment;

FIG. 2 is a diagram illustrating a process of pre-training a graph neural network (GNN) based on a molecular descriptor according to an example embodiment;

FIG. 3 is a diagram illustrating an example of a molecular descriptor according to an example embodiment;

FIG. 4 is a diagram illustrating a method of training a prediction model for performing a target task according to an example embodiment;

FIG. 5 is a flowchart illustrating a method of predicting a target task according to an example embodiment;

FIG. 6 is a diagram illustrating a method of predicting a target task using a prediction model according to an example embodiment; and

FIG. 7 is a block diagram illustrating an apparatus for predicting a target task according to an example embodiment.

### DETAILED DESCRIPTION

The following detailed structural or functional description of embodiments is provided as an example only and various alterations and modifications may be made to the embodiments. Here, the embodiments are not construed as limited to the disclosure and should be understood to include all changes, equivalents, and replacements within the idea and the technical scope of the disclosure.

Although terms, such as first, second, and the like are used to describe various components, the components are not limited to the terms. These terms should be used only to distinguish one component from another component. For example, a first component may be referred to as a second component, and similarly the second component may also be referred to as the first component.

It should be noted that when a component or element is described as being “connected to”, “coupled to”, or “joined to” another component or element, it may be directly (e.g., in contact with the other component or element) “connected to”, “coupled to”, or “joined to” the other component or element, or there may reasonably be one or more other components or elements intervening therebetween.

The singular forms “a”, “an”, and “the” are intended to include the plural forms as well, unless the context clearly indicates otherwise. It will be further understood that the terms “comprises/comprising” and/or “includes/including” when used herein, specify the presence of stated features, numbers, operations, members, elements, and/or combinations thereof, but do not preclude the presence or addition of one or more other features, numbers, operations, members, elements, and/or combinations thereof.

Unless otherwise defined, all terms, including technical and scientific terms, used herein have the same meaning as commonly understood by one of ordinary skill in the art to which the present disclosure pertains. Terms, such as those defined in commonly used dictionaries, are to be interpreted as having a meaning that is consistent with their meaning in the context of the relevant art, and are not to be interpreted in an idealized or overly formal sense unless expressly so defined herein.

Hereinafter, the embodiments will be described in detail with reference to the accompanying drawings. When describing the embodiments with reference to the accompanying drawings, like reference numerals refer to like components and a repeated description related thereto will be omitted.

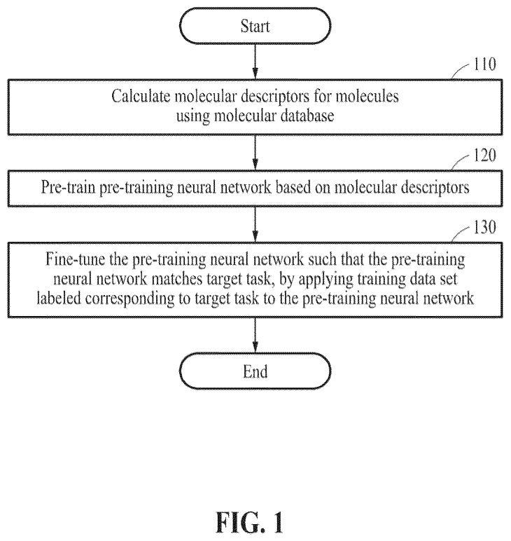

FIG. 1 is a flowchart illustrating a method of training a prediction model for performing a target task according to an example embodiment. In the following example embodiments, operations may be sequentially performed, but not necessarily performed sequentially. For example, the order of the operations may be changed and at least two of the operations may be performed in parallel.

Referring to FIG. 1, an apparatus (hereinafter, referred to as a “training apparatus”) for training a prediction model may train a prediction model through operations 110, 120, and 130.

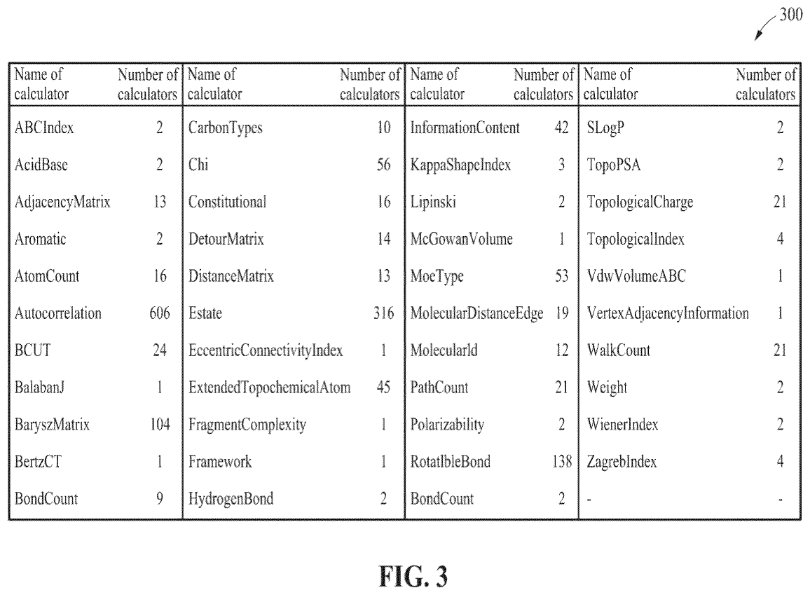

In operation 110, the training apparatus may calculate molecular descriptors for molecules using a molecular database. The training apparatus may use software that calculates a molecular descriptor, for example, a Mordred molecular descriptor calculator 300 shown in FIG. 3, to calculate the molecular descriptors for the molecules. However, embodiments are not limited thereto. The molecular descriptors may be, for example, two-dimensional (2D) molecular descriptors.

In operation 120, the training apparatus may pre-train a pre-training neural network based on the molecular descriptors calculated in operation 110. For example, the training apparatus may reduce dimensionality of the molecular descriptors using a principal component analysis (PCA). In this example, the PCA may be used to find principal components of distributed data. The PCA may be used to analyze a principal component of one distribution when multiple pieces of data are collected together to form a distribution, instead of analyzing a component of each piece of data. The principal component may be a direction vector corresponding to a direction with a largest variance of data in one distribution. For example, when the PCA is performed on a set of 2D data, two principal component vectors perpendicular to each other may be output. When the PCA is performed on three-dimensional (3D) points, three principal component vectors perpendicular to each other may be output. The training apparatus may generate a pre-training data set including molecular graphs corresponding to molecules and first latent vectors corresponding to the molecular graphs by reducing the dimensionality of the molecular descriptors using the PCA.

For example, the training apparatus may set the number of principal component vectors so that 70% of the total variance is obtained and explained.

The training apparatus may simplify an output representation by removing redundant information (e.g. a linear dependency) between molecular descriptors in a process of reducing the dimensionality. Each prediction target may be standardized to, for example, an average of “0” and a variance of “1” for the training data set.

The training apparatus may pre-train the pre-training neural network using the molecular descriptors with the reduced dimensionality as pseudo labels of the molecular graphs. The pre-training neural network may include, for example, at least one of a graph neural network (GNN) or a large language model (LLM), but is not necessarily limited thereto.

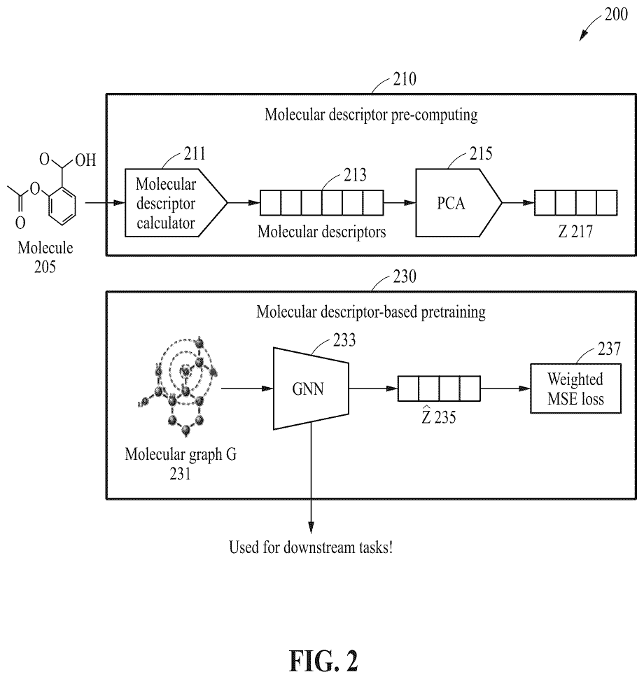

The training apparatus may assign the first latent vectors to the pseudo labels of the molecular graphs respectively corresponding to the molecules. Based on the pseudo labels, the training apparatus may pre-train the pre-training neural network to predict a target pseudo label (e.g., a second latent vector) corresponding to a target molecule. A method by which the training apparatus pre-trains the pre-training neural network will be described in more detail with reference to FIG. 2 below.

In operation 130, the training apparatus may adjust and fine-tune the pre-training neural network such that the pre-training neural network may match the target task by applying a training data set labeled corresponding to the target task to the pre-training neural network that is pre-trained in operation 120.

The target task may include, for example, at least one of a prediction of a yield of a target chemical reaction corresponding to the target task, a prediction of a reaction condition of the target chemical reaction, or a prediction of physical properties of the target chemical reaction, but is not limited thereto. In addition, the target task may include various tasks that may be predicted from a molecular descriptor and/or structural information of a molecule. In an example, the training data set labeled corresponding to the target task may be a training data set labeled with a target chemical reaction corresponding to the target task and a target yield corresponding to the target chemical reaction. In another example, the training data set labeled corresponding to the target task may be a training data set labeled with a target chemical reaction corresponding to the target task and target physical properties or a target reaction condition corresponding to the target chemical reaction.

The training data set labeled corresponding to the target task may be specialized for each of various application fields, such as medicine, electronic materials, and/or semiconductors, and may be provided in advance. The training data set labeled corresponding to the target task may be, for example, a commercial database such as ChEMBL, ZINC-subset, and PubChem, but is not necessarily limited thereto.

In an example embodiment, to enhance performance of a prediction model in a scenario in which a diversity of the training data set is insufficient despite a sufficient quantity of the training data set, a prediction model that predicts a target task may be trained through a 3-phase procedure that will be described below. Accordingly, yield prediction performance may be enhanced for a situation in which training data is insufficient or for a chemical reaction that is absent in training data.

First, the training apparatus may define a pre-text task based on molecular descriptors, using a large-scale molecular database. The pre-text task may correspond to a process of obtaining and calculating the above-described molecular descriptors through operation 110. Subsequently, the training apparatus may pre-train a pre-training neural network (e.g., a GNN) as in operation 120 using molecular descriptors obtained from the pre-text task. Finally, the training apparatus may integrate the pre-trained pre-training neural network as a portion of the prediction model and fine-tune the prediction model using the training data set as in operation 130.

FIG. 2 is a diagram illustrating a process of pre-training a GNN based on a molecular descriptor according to an example embodiment. Referring to FIG. 2, a diagram 200 illustrates a process in which a training apparatus pre-trains a GNN according to an example embodiment.

The training apparatus may pre-train the GNN using a relatively large-scale molecular database and perform a target task such as a prediction of a yield of a chemical reaction, using the pre-trained GNN, thereby providing a high-performance prediction model even with a relatively small quantity of data while overcoming a reduction in performance of a material-based prediction model with an insufficient quantity of training data or insufficient diversity.

The GNN may be analyzed to be more effective in predicting a yield of a chemical reaction. However, when the quantity of training data or the diversity of the training data is insufficient, performance may tend to decrease even when the GNN is trained using the training data set.

Here, the chemical reaction may be a process in which a reactant is changed to a product through a chemical change or deformation. In addition, the yield of the chemical reaction may be expressed as a percentage of the amount of a product generated in comparison to a consumed reactant. Since a prediction of a yield of a chemical reaction provides a clue to a search for a high-yield chemical reaction without direct experimental measurements, the time and cost used in a development process may be significantly reduced.

The training apparatus may perform a molecular descriptor pre-computing process 210, and a molecular descriptor-based pre-training process 230.

In the molecular descriptor pre-computing process 210, when a molecular database including a large number of molecules is provided, the training apparatus may calculate molecular descriptors 213 corresponding to each of the molecules using a molecular descriptor 211. The training apparatus may generate molecular descriptors (e.g., first latent vectors Z 217) with a reduced dimensionality by applying a PCA 215 to the molecular descriptors 213. Here, the training apparatus may assign a vector of a principal component score calculated through the PCA 215 for the molecular descriptors 213 as a pseudo label to each of the molecules included in the molecular database.

In the molecular descriptor-based pre-training process 230, the training apparatus may pre-train a pre-training neural network (e.g., a GNN 233) to perform a pre-text task of predicting a pseudo label for an input molecule 205. In the pre-text task, a new graph-level pre-text task for pre-training of the GNN 233 may be defined by using a molecular descriptor as a prediction target. The molecular descriptor-based pre-training process 230 may be performed for each downstream task.

The training apparatus may input a target molecular graph G 231 corresponding to the input molecule 205 representing structural information of a target molecule to the pre-training neural network (e.g., the GNN 233) and output a second latent vector \hat{z} 235 that is a molecular representation vector corresponding to the target molecular graph G 231. For example, the GNN 233 may receive the molecular graph G 231 as an input and predict the second latent vector \hat{z} 235 that is a predicted value of a free-text task such as \hat{z}=f(G).

The training apparatus may train the GNN 233 such chat a difference between the calculated molecular descriptors (e.g., the first latent vectors 217) and the second latent vector 235 based on an output of the GNN 233 may be minimized. The training apparatus may train the GNN 233, for example, based on the difference between the first latent vectors 217 and the second latent vector 235. For example, the training apparatus may use a loss function 237 (e.g., an objective function based on a weighted mean square error (WMSE) loss between the first latent vectors 217 and the second latent vector 235). The training apparatus may train the GNN 233 as in L(z,\hat{z})=(z−\hat{z})^{T}Λ(z−\hat{z}) by a WMSE loss using a square root of an eigenvalue of each target task that is to be predicted as a weight of a corresponding target task. Here, a k-th diagonal element λₖ of a diagonal matrix Λ∈ [symbol] ^{d×d} may correspond to a square root of an eigenvalue of a k-th target task that is to be predicted. The square root of the eigenvalue may correspond to a standard deviation of the target task.

For example, when a pre-training data set D={Gₜ,zₜ}ₜ₌₁^{N} is provided, the training apparatus may train the GNN 233 to minimize an objective function

$$𝒥 = \frac{1}{N} {\sum }_{t = 1}^{N} L ( z , \hat{z} ) .$$

Subsequently, a prediction apparatus according to an example embodiment may initialize the prediction model using the GNN 233 pre-trained to predict a yield of a chemical reaction, and may fine-tune the GNN 233 using a training data set including a chemical reaction and a yield.

FIG. 3 is a diagram illustrating an example of a molecular descriptor according to an example embodiment. FIG. 3 illustrates the Mordred molecular descriptor calculator 300 according to an example embodiment.

It may be difficult to secure a sufficient quantity of training data generated through an experiment for machine learning due to time and/or cost, and biased data may also be accumulated depending on predetermined conditions. In addition, the physical properties or synthesis direction may be often different even when structural expressions of molecules are very similar. Here, a cold-start problem in which a prediction model is overfitted may occur due to a small quantity of data.

In an example embodiment, a pre-training neural network may be pre-trained using a relatively large amount of molecular structures and molecular descriptors that may be more easily calculated, and accordingly, a lack of training data or biased training data when a prediction model is implemented may be supplemented.

In addition, in an example embodiment, the prediction model may be allowed to learn more accurate descriptors, using a molecular structure together with a molecular descriptor in training of the pre-training neural network, to more accurately perform various target tasks such as a prediction of physical properties, a prediction of a reaction yield, and/or a prediction of a synthesis condition.

The Mordred molecular descriptor calculator 300 may be, for example, a Python package and may correspond to software that calculates molecular descriptors that may represent quantitative structures and property relationships. The molecular descriptors may be used to represent various molecular properties.

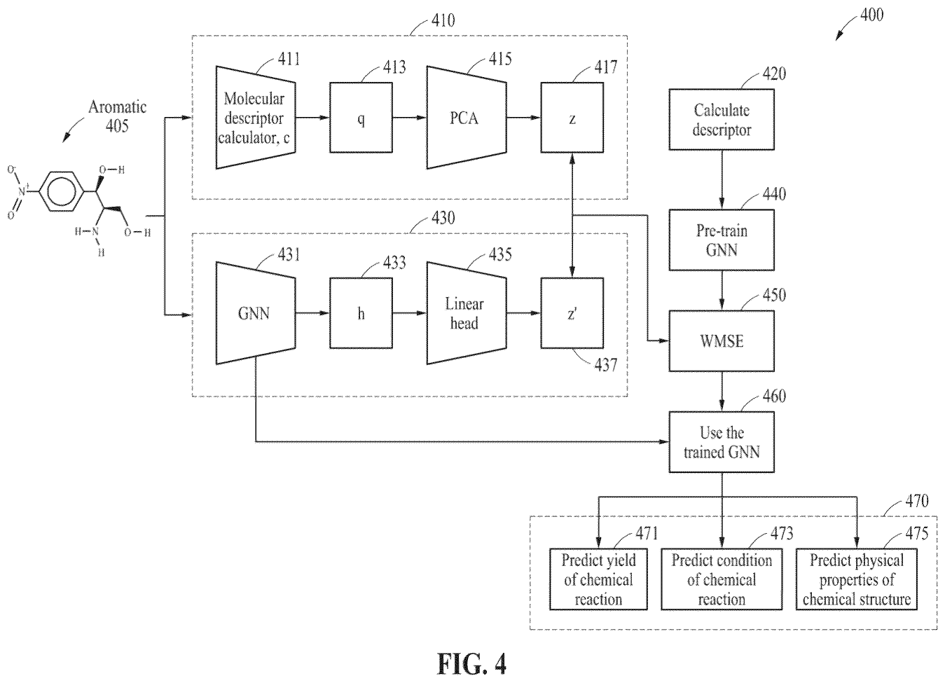

FIG. 4 is a diagram illustrating a method of training a prediction model for performing a target task according to an example embodiment. Referring to FIG. 4, a diagram 400 illustrates a process of predicting a target task through a process of pre-training a molecular descriptor-based GNN according to an example embodiment.

A training apparatus according to an example embodiment may include a PCA-based calculator PCA model 410, and a molecular structure-based GNN model 430.

When a molecular graph 405 representing structural information of a target molecule is input, the training apparatus may output a molecular descriptor 420 calculated through the calculator PCA model 410. The molecular descriptor 420 may be, for example, “1613” 2D molecular descriptors based on a MORDRED molecular descriptor calculator.

An example of an operation of the calculator PCA model 410 is described below.

In an example embodiment, a molecular descriptor 413 may be used to perform a pre-text task to pre-train a GNN 431.

The training apparatus may obtain a pseudo label by performing a pre-text task corresponding to the molecular graph 405 through the calculator PCA model 410. The molecular descriptor 413 included in dimensionality reduced through a PCA may be used as a pseudo label for the input molecule 405. A molecular descriptor may be a numerical representation of chemical information of a molecule derived through logical and mathematical procedures, that is, a result obtained by converting various chemical features included in a molecule into numerical values. In general, a molecular descriptor 411 may be mainly used in the form of input data of a molecule in a wide range of tasks of predicting physical properties of molecules.

For example, when a large molecular data set [symbol] ={ [symbol] ᵢ}ᵢ₌₁^{M} is provided, the training apparatus may calculate molecular descriptors q, 413 using a Mordred molecular descriptor calculator c 411. For example, the Mordred molecular descriptor calculator c 411 may generate up to “1,826” molecular descriptors 413 per molecule. The molecular descriptors qᵢ 413 may be efficiently calculated at a high speed with high scalability for large molecules.

For example, the training apparatus may calculate molecular descriptors qᵢ∈ [symbol] ^{d} 413 for each molecule [symbol] ᵢ, as shown in Equation 1 below.

$${q}_{i} = c ( {𝒢}_{i} ) , ∀ i \qquad [ Equation 1 ]$$

The molecular descriptors qᵢ∈ [symbol] ^{d} 413 may be high-dimensional and may include redundant information.

The training apparatus may use a PCA 415 to reduce a dimension of a vector while maintaining original information at the maximum level to remove redundant information. The training apparatus may generate new features formed through a linear combination of the original molecular descriptors, through the PCA 415, may allow the new features to describe a largest change in molecular descriptors, and may ensure that the new features are unrelated to each other.

The training apparatus may use the PCA 415 to remove redundant information from the molecular descriptors qᵢ 413. The training apparatus may generate new features by an eigendecomposition of a covariance matrix S of the molecular descriptors qᵢ 413 and may ensure that the new features are unrelated to each other.

The training apparatus may obtain eigenvectors u₁, . . . , u_{b} of highest “b” maximum eigenvalues λ₁, . . . , λ_{b} from the covariance matrix s of a set {qᵢ}ᵢ₌₁^{M} of the molecular descriptors qᵢ 413. The eigendecomposition of the covariance matrix may correspond to the eigenvalues λ₁, . . . , λ_{b}, and the training apparatus may calculate “b” eigenvectors called “principal components.”

The training apparatus may reduce dimensionality by projecting each of the molecular descriptors qᵢ 413 to an eigenspace through the eigenvectors u₁, . . . , u_{b} to obtain a q-dimensional vector (q<p) reduced through the PCA, thereby obtaining first latent vectors Z 417 corresponding to the molecular descriptors with the reduced dimensionality, as shown in Equation 2 below.

$$\begin{matrix}{z}_{i} = ( {z}_{i 1} , … , {z}_{i b} ) \\ = ( {u}_{i}^{T} {q}_{i} , … , {u}_{b}^{T} {q}_{i} ) , ∀ i ; b < d\end{matrix} \qquad [ Equation 2 ]$$

Here, a latent vector zᵢ may correspond to a principal component score of an eigenvector obtained using an i-th principal component. Each latent vector zᵢ may be assigned as a pseudo label to a corresponding molecular graph [symbol] ᵢ 405, and the pre-text task may be performed.

When the first latent vectors z 417 are obtained, the training apparatus may generate a pre-training data set \tilde{D}={ [symbol] ᵢ,zᵢ}ᵢ₌₁^{M}. The pre-training data set may match molecular descriptors (e.g., the first latent vectors z 417) that have the reduced dimensionality and that respectively correspond to the molecular graphs [symbol] ᵢ 405.

The training apparatus may input the molecular graphs [symbol] ᵢ 405 to the GNN model 430, to perform pre-training 440 of the GNN 431.

In a pre-training operation by the GNN model 430, the training apparatus may input the molecular graphs [symbol] ᵢ 405 to the GNN 431 and output a molecular representation vector hᵢ 433.

The training apparatus may use a graph isomorphism network (GIN) as a backbone of the GNN 431, and in particular, may apply a variant of a GIN that integrates edge features with an input representation.

The node embedding size of the GNN 431 may be “256”, but is not necessarily limited thereto. The GNN 431 may use, for example, five layers. The training apparatus may perform layer normalization, graph size normalization, and/or residual connection for each layer of the GNN 431. Readout in units of graphs may be performed, for example, by multi-layer perceptron (MLP) readout including at least one non-linear hidden layer.

The training apparatus may use a linear head 435 to process a graph-level molecular representation vector h 433 to obtain a second latent vector \hat{z} 437 that is a predicted value of a pseudo label (e.g., the first latent vector z 417). Here, the linear head 435 may be used in the pre-training process only, and may not be used in a subsequent prediction process.

For example, the GNN 431 may process an input molecular graph [symbol] =( [symbol] , [symbol] ), which is described below.

The GNN 431 may use edge embedding functions ϕₙ and ϕₑ embed each of node vectors v^{j}∈ [symbol] and each of edge vectors e^{j,k}∈ [symbol] to an initial node hᵥ^{j,(0)} and edge embedding hₑ^{j,k}, as shown in Equations 3 and 4 below.

$${h}_{v}^{j , ( 0 )} = {ϕ}_{n} ( {v}^{j} ) ; \qquad [ Equation 3 ]$$

$${h}_{e}^{j , k} = {ϕ}_{e} ( {e}^{j , k} ) . \qquad [ Equation 4 ]$$

In Equations 3 and 4, ϕₙ and ϕₑ may be parameterized with a neural network.

The GNN 431 may aggregate information of neighboring nodes using “L” message passing layers and repeatedly update node embeddings. In an L-th layer (l=1, . . . , L), each node embedding hᵥ^{j,(l)} may be updated as shown in Equation 5 below.

$${h}_{v}^{j , ( l )} = {ψ}^{( l )} ( {h}_{v}^{j , ( l - 1 )} + {\sum }_{k \vert {e}^{j , k} ∈ ℰ} Re LU ( {h}_{v}^{j , ( l - 1 )} + {h}_{e}^{j , k} ) ) . \qquad [ Equation 5 ]$$

In Equation 5, ψ^{(l)} may correspond to an l-th node embedding function parameterized with the GNN 431. Here, ReLU denotes a ReLU activation function.

The GNN 431 may combine final node embeddings hᵥ^{j,(L)} through an average pooling and extract a graph embedding vector h_{g} as shown in Equation 6 below.

$${h}_{g} = \frac{1}{\vert 𝒱 \vert} {\sum }_{j \vert {v}^{j} ∈ 𝒱} {h}_{v}^{j , ( L )} . \qquad [ Equation 6 ]$$

The GNN 431 may obtain the molecular representation vector h 433 as shown in Equation 7 below, by processing a graph embedding h_{g} by a projection function r. The molecular representation vector h 433 may correspond to a graph-level molecular representation vector.

$$h = r ( {h}_{g} ) \qquad [ Equation 7 ]$$

The training apparatus may predict the second latent vector \hat{z}ᵢ=(\hat{z}ᵢ₁, . . . , \hat{z}_{ib}) 437 corresponding to a prediction result of the first latent vector zᵢ 417 by inputting the molecular representation vector hᵢ 433 to the linear head 435. The second latent vector \hat{z}ᵢ 437 may correspond to a target pseudo label of each molecular graph [symbol] ᵢ 405.

Here, the linear head 435 may be, for example, an MLP including “512” ReLU units with three layers, and may be batch normalized. A dropout rate of the linear head 435 may be, for example, “0.1,” but is not necessarily limited thereto. The linear head 435 may be used in only the pre-training operation by the GNN model 430.

The GNN 431 and the linear head 435 may be simultaneously trained by minimizing an objective function \tilde{ℑ} based on a WMSE between the first latent vector zᵢ 417 and the second latent vector \hat{z}ᵢ 437 using eigenvalues λ as shown in Equation 8 below. Equation 8 may correspond to a J loss function calculation formula that calculates a mean square error (MSE) value.

$$\tilde{𝒥} = \frac{1}{M \cdot b} {\sum }_{i = 1}^{M} {\sum }_{i = 1}^{b} {{λ}_{j} ( {z}_{ij} - {\hat{z}}_{ij} )}^{2} \qquad [ Equation 8 ]$$

In Equation 8, M and b denote the number of pieces of data. i and j denote indices of a 2D vector (i, j). In addition, zᵢⱼ denotes the first latent vector zᵢ 417, and \hat{z}ᵢⱼ denotes the second latent vector \hat{z}ᵢ 437.

For example, when a pre-training data set [symbol] ={( [symbol] ᵢ, zᵢ)}ᵢ₌₁^{M} for a pre-text task is provided, the training apparatus may train the GNN 431 and the linear head 435 together using a loss function [symbol] defined as in Equation 9 below. Equation 9 may correspond to an MSE calculation formula that estimates a loss value for training of a prediction model.

$$\tilde{ℒ} ( z , \hat{z} ) = \frac{1}{q} {\sum }_{j = 1}^{q} {{λ}_{j} ( {z}_{j} - {\hat{z}}_{j} )}^{2} , \qquad [ Equation 9 ]$$

In Equation 9, λⱼ denotes an eigenvalue obtained by the PCA 415, and q denotes the number of pieces of data.

The training apparatus may calculate a WMSE 450 between the predicted second latent vector \hat{z}ᵢ 437 and the first latent vector zᵢ 417 corresponding to the output of the calculator PCA model 410, and may perform back propagation on the molecular structure-based GNN model 430. In addition, the training apparatus may downstream the pre-trained GNN 431 and utilize the GNN 431 in operation 460 to perform a prediction 471 of a yield of a chemical reaction, a prediction 473 of a chemical reaction condition, and/or a prediction 475 of physical properties of a chemical structure in a prediction model 470.

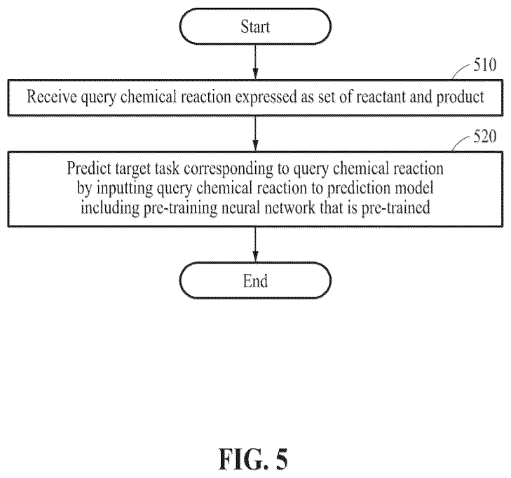

FIG. 5 is a flowchart illustrating a method of predicting a target task according to an example embodiment. In the following example embodiments, operations may be performed sequentially, but not necessarily performed sequentially. For example, the order of the operations may be changed and at least two of the operations may be performed in parallel.

Referring to FIG. 5, an apparatus for predicting a target task (hereinafter, referred to as a “prediction apparatus”) may predict a target task through operations 510 and 520.

In operation 510, the prediction apparatus may receive a query chemical reaction expressed as a set of a reactant and a product. Here, the query chemical reaction may correspond to a target chemical reaction on which a target task, such as a prediction of a yield, a prediction of physical properties, or a prediction of a synthesis condition, is to be performed, that is, correspond to a chemical reaction input to the prediction apparatus.

In operation 520, the prediction apparatus may predict a target task corresponding to the query chemical reaction by inputting the query chemical reaction received in operation 510 to a prediction model including a pre-training neural network that is pre-trained. Here, the pre-training neural network may be the pre-training neural network that is pre-trained through the process described above with reference to FIGS. 1 to 4. The prediction model may be fine-tuned to predict a result matching the target task by applying a training data set labeled corresponding to the target task to the pre-training neural network.

The prediction model may predict a yield corresponding to the query chemical reaction when the query chemical reaction expressed as the set of the reactant and the product is input. The reactant may include molecular graphs representing a plurality of reactant molecules corresponding to different reactions, and the product may include a single molecular graph representing a product molecule.

For example, a single molecule may be represented by an undirected graph G=(V, E). In this example, V may be a set of nodes associated with heavy atoms in one molecule. E may be a set of edges associated with a chemical bond between heavy atoms.

The molecular graphs and single molecular graph may each include node vectors representing node features corresponding to heavy atoms in a molecule, and edge vectors representing edge features corresponding to chemical bonds between the heavy atoms in the molecule. The node features may include, for example, at least one of an atom type of the heavy atoms, formal charges of the heavy atoms, a degree of the heavy atoms, a hybridization of the heavy atoms, a number of atoms adjacent to the heavy atoms, a valence of the heavy atoms, a chirality of the heavy atoms, associated ring sizes of the heavy atoms, whether the heavy atoms donate or accept electrons, whether the heavy atoms are aromatic, or whether the heavy atoms include a ring.

The edge features may include at least one of a bond type of the chemical bonds between the heavy atoms, a stereochemistry of the chemical bonds between the heavy atoms, whether a ring is in the chemical bonds between the heavy atoms, or whether the chemical bonds between the heavy atoms are conjugated.

Here, the bond type of the chemical bonds may be the type of a bond or force exerted between constituent atoms in an atom aggregate. The bond type may include, for example, a covalent bond, an ionic bond, a hydrogen bond, a metallic bond, and a coordinate covalent bond, a van der Waals force (dispersion force) bond, and a hydrophobic bond, but is not necessarily limited thereto. The covalent bond may be a bonding state in which two atoms share a pair of electrons in an orbital. The ionic bond may refer to a bond that gains or loses electrons between a cation and an anion and that is formed by an electrostatic attraction. The hydrogen bond may refer to a bond between hydrogen (H) and fluorine (F), oxygen (O) and nitrogen (N) which have high electronegativity. The metallic bond may be a bond caused by an electrical attraction between electrons and ions evenly distributed in a metal. The metallic bond may be, for example, a chemical bond that provides various properties of metals, such as strength, malleability, ductility, luster, thermal conductivity, and electrical conductivity. The coordinate covalent bond may refer to a bond in which electrons involved in the bond are formally provided only by one atom when two atoms form a covalent bond. The van der Waals force bond may refer to a bond formed when electrons are concentrated locally within a nonpolar molecule and become charged and when an attractive force is exerted between molecules. A hydrophobic interaction force may be a force generated between nonpolar molecules in water, and water molecules may be aligned around a hydrophobic portion of a molecule due to the hydrophobic interaction force.

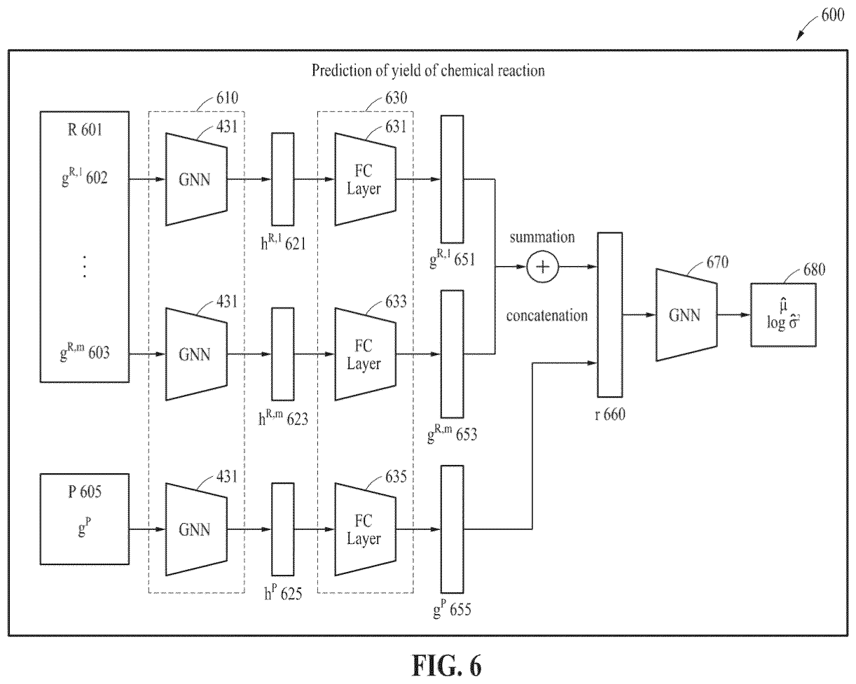

The stereochemistry may be a 3D structure of a molecule or a phenomenon associated with the 3D structure, and involve a spatial arrangement of atomic groups or atoms included in the molecule in 3D. A conjugation may indicate that a single bond and a double bond (or a multiple bond) are alternately connected, for example, in benzene. A structure and an operation of the prediction model are described in detail with reference to FIG. 6 below.

FIG. 6 illustrates a structure and an operation of a prediction model according to an example embodiment. Referring to FIG. 6, a structure of a prediction model 600 according to an example embodiment is illustrated.

The prediction model 600 may include at least one pre-training neural network 610, at least one fully-connected layer 630, and a feedforward neural network (FNN) 670.

The at least one pre-training neural network 610 may process a query molecular graph (e.g., molecular graphs [symbol] 602, 603, and 605) within a query chemical reaction and may output molecular query representation vectors (e.g., molecular representation vectors h 621, 623, and 625). The at least one pre-training neural network 610 may correspond to, for example, the GNN 431 described above with reference to FIG. 4, but is not necessarily limited thereto.

The at least one fully-connected layer 630 may respectively correspond to the at least one pre-training neural network 610 and may output high-dimensional molecular representation vectors g 651, 653, and 655 corresponding to the molecular query representation vectors.

The FNN 670 may integrate the high-dimensional molecular representation vectors g 651, 653, and 655 and output a prediction result corresponding to a target task by a representation vector r 660 of a chemical reaction calculated from the integrated high-dimensional molecular representation vectors g 651, 653, and 655. The prediction result may include, for example, a predicted average and predicted log variance from the representation vector r 660 of the chemical reaction, but is not necessarily limited thereto. An FNN may also be a prediction head.

The prediction model 600 may further include a one-hot-encoding layer corresponding to each of a temperature condition, a pressure condition, and a solvent condition corresponding to a query chemical reaction. Here, the one-hot-encoding layer may be positioned between the at least one fully-connected layer 630 and the FNN 670.

For example, the prediction model 600 that uses a chemical reaction ( [symbol] , [symbol] ) 601 and 605 as an input to predict a yield y of the chemical reaction ( [symbol] , [symbol] ) may be configured. The prediction model 600 may be trained by a training data set to [symbol] ={( [symbol] ᵢ, [symbol] ᵢ, yᵢ)}ᵢ₌₁^{N} to predict a yield y of a chemical reaction that is a target task.

When a query chemical reaction ( [symbol] _{*}, [symbol] _{*}) is provided, a prediction apparatus may predict a yield y_{*} using the prediction model 600, as shown in Equation 10 below.

$${\hat{y}}_{*} = f ( {ℛ}_{*} , {𝒫}_{*} ) . \qquad [ Equation 10 ]$$

Here, a data representation used in the prediction model 600 is described. Each chemical reaction may be expressed as, for example, (R, P, y). Here, R 601 denotes a reactant set, P 605 denotes a product set, and y denotes a yield of a chemical reaction.

The reactant set [symbol] ={ [symbol] [symbol] ^{, 1}, . . . , [symbol] [symbol] ^{, m}} 601 may include “m” reactant molecules represented by molecular graphs. Here, “m” may vary depending on chemical reactions. The product set [symbol] ={ [symbol] [symbol] } 605 may include a single molecular graph representing a product molecule. In each molecular graph [symbol] =( [symbol] ), [symbol] may represent a set of nodes associated with heavy atoms, and [symbol] may represent a set of edges associated with chemical bonds between nodes.

For example, a hydrogen atom may implicitly be processed by node features of neighboring heavy atoms. Each node vector may represent a node feature of a j-th heavy atom in a molecule. The node feature may include, for example, an atom type of the j-th heavy atom, formal charge of the j-th heavy atom, a degree of the j-th heavy atom, a hybridization of the j-th heavy atom, the number of adjacent hydrogens, a valence of the j-th heavy atom, a chirality of the j-th heavy atom, associated ring sizes of the j-th heavy atom, whether the j-th heavy atom donates or accepts electrons, whether the j-th heavy atom is aromatic, or whether the j-th heavy atom includes a ring. Each edge vector may represent an edge feature associated with a chemical bond between the j-th heavy atom and a k-th heavy atom. The edge feature may include, for example, a bond type of the chemical bond, a stereochemistry of the chemical bond, whether a ring is in the chemical bond, or whether chemical bonds are conjugated.

In an example embodiment, a GIN structure may be used as an element forming a GNN of the prediction model 600 to predict a yield of a chemical reaction.

The prediction apparatus may initialize the at least one pre-training neural network 610 using a pre-trained parameter θ obtained from a previous operation to use prior knowledge in a pre-text task.

For example, p_{θ}(y| [symbol] ) may be assumed to follow a normal distribution of an average μ and a variance σ². In this example, the prediction model f 600 may receive the chemical reaction ( [symbol] ) 601 and 605 for an estimation of p_{θ} and may output a predicted average \hat{μ} and a predicted variance \hat{σ}² (or log \hat{σ}²) 680 for a yield y through the parameter θ, as shown in Equation 11 below.

$$( \hat{μ} , log {\hat{σ}}^{2} ) = f ( ℛ , 𝒫 ; θ ) \qquad [ Equation 11 ]$$

The prediction model 600 may include the at least one pre-training neural network 610 to obtain representation vectors of molecules in the chemical reaction ( [symbol] ) and one FNN 670 to return the final output.

The at least one pre-training neural network 610 may generate the molecular representation vectors h 621, 623, and 625 respectively corresponding to the molecular graphs [symbol] 602, 603, and 605 by receiving the molecular graphs [symbol] 602, 603, and 605 in the chemical reaction ( [symbol] ).

The prediction apparatus may input the molecular representation vectors h 621, 623, and 625 respectively corresponding to the molecular graphs [symbol] 602, 603, and 605 to one layer of the at least one fully-connected layer (FC Layer) 630, and may expand the molecular representation vectors h 621, 623, and 625 as new high-dimensional molecular representation vectors g 651, 653, and 655.

Accordingly, the prediction apparatus may obtain molecular representation vector sets {g [symbol] ^{,1}, . . . , g [symbol] ^{,m}} 651 and 653 corresponding to the reactant set [symbol] 601, and a molecular representation vector set {g [symbol] } 655 corresponding to the product set [symbol] 605.

The prediction apparatus may sum the molecular representation vector sets {g [symbol] ^{,1}, . . . , g [symbol] ^{,m}} 651 and 653 corresponding to the reactant set [symbol] 601, may concatenate a sum of the molecular representation vector sets {g [symbol] ^{,1}, . . . , g [symbol] ^{,m}} 651 and 653 to the molecular representation vector g [symbol] 655 corresponding to the product set [symbol] 605, and may calculate the representation vector r 660 of the chemical reaction as shown in Equation 12 below.

$$r = [ {\sum }_{l = 1}^{m} {g}^{ℛ , l} , {g}^{𝒫} ] \qquad [ Equation 12 ]$$

The representation vector r 660 of the chemical reaction may be finally input to the FNN 670, and the FNN 670 may output the predicted average \hat{μ} and the predicted log variance log \hat{σ}² 680.

The FNN 670 may perform a final prediction by integrating all molecular representation vectors. A parameter of each component of the at least one pre-training neural network 610 for training of the prediction model 600 may be initialized using the pre-trained GNN 431 and the other parameters may be randomly initialized.

For example, a training data set [symbol] ={( [symbol] ᵢ, [symbol] ᵢ,yᵢ)}ᵢ₌₁^{N} for a target task including “N” chemical reactions and yields thereof may be provided.

In this example, the prediction model 600 may be fine-tuned using a loss function [symbol] as shown in Equation 13 below.

$$ℒ ( y , \hat{μ} , {\hat{σ}}^{2} ) = ( 1 - α ) {( y - \hat{μ} )}^{2} + α [ \frac{{( y - \hat{μ} )}^{2}}{{\hat{σ}}^{2}} + log {\hat{σ}}^{2} ] , \qquad [ Equation 13 ]$$

In Equation 13, a first term (1−α)(y−\hat{μ})² and a second term

$$α [ \frac{{( y - \hat{μ} )}^{2}}{{\hat{σ}}^{2}} + log {\hat{σ}}^{2} ]$$

may be associated with a loss under homoscedastic and heteroscedastic assumptions, respectively. In addition, α denotes a hyperparameter that controls a relative strength of the above two terms, \hat{μ} denotes a predicted average, and y denotes a yield to be predicted.

When the training data set [symbol] ={ [symbol] ᵢ, [symbol] ᵢ,yᵢ}ᵢ₌₁^{N} is provided, the prediction model 600 may be trained while minimizing an objective function ℑ based on the loss function [symbol] , for example, as shown in Equation 14 below.

$$𝒥 = \frac{1}{N} {\sum }_{i = 1}^{N} ℒ ( {y}_{i} , {\hat{μ}}_{i} , log {\hat{σ}}^{2} ) \qquad [ Equation 14 ]$$

In Equation 14, N denotes the number of pieces of data.

Here, the pre-trained GNN 431 may have, as initial values, parameters pre-trained based on molecular descriptors, and may be fine-tuned and used to predict a yield of a chemical reaction according to the training of the prediction model 600.

When a new chemical reaction ( [symbol] _{*}, [symbol] _{*}) is provided, the trained prediction model 600 may predict a yield y_{*}.

For example, the prediction model 600 may obtain “T” prediction results {(\hat{μ}_{*}^{(t)},log \hat{σ}_{*}^{2(t)})}ₜ₌₁^{T} based on a Monte-Carlo (MC) dropout, to acquire a final predicted yield ŷ_{*} as shown in Equation 15 below.

$${\hat{y}}_{*} = \frac{1}{T} {\sum }_{t = 1}^{T} {\hat{μ}}_{*}^{( t )} \qquad [ Equation 15 ]$$

In Equation 15, t denotes the number of times training is performed from “1” to the total number T of pieces of training data.

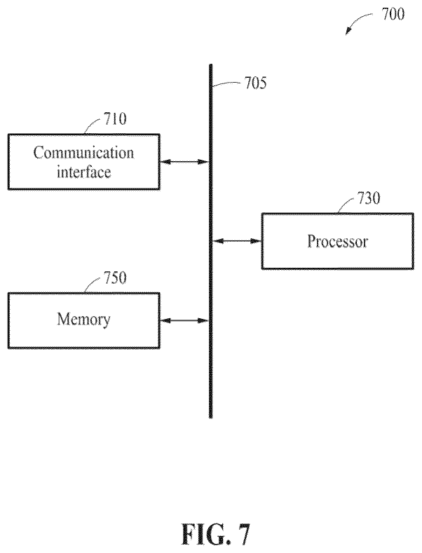

FIG. 7 is a block diagram illustrating an apparatus for predicting a target task according to an embodiment. Referring to FIG. 7, a prediction apparatus 700 may include a communication interface 710, a processor 730, and a memory 750. The communication interface 710, the processor 730, and the memory 750 may communicate with each other via a communication bus 705.

The communication interface 710 may receive a query chemical reaction expressed as a set of a reactant and a product.

The processor 730 may predict a yield corresponding to the query chemical reaction received by the communication interface 710, by inputting the query chemical reaction to a prediction model including a pre-trained GNN. The prediction model may be fine-tuned to predict the yield corresponding to the query chemical reaction by applying a training data set labeled corresponding to the predicted yield to the GNN.

The memory 750 may store a variety of information generated in the processing process of the processor 730 described above. In addition, the memory 750 may store a variety of data and programs. The memory 750 may be, for example, a volatile memory or a non-volatile memory. The memory 750 may include a large-capacity storage medium such as a hard disk to store a variety of data.

In addition, the processor 730 may perform at least one of the methods described with reference to FIGS. 1 to 6 or an algorithm corresponding to at least one of the methods. The processor 730 may be a hardware-implemented data processing device including a circuit having a physical structure to perform desired operations. The desired operations may include, for example, code or instructions included in a program. The processor 730 may be implemented as, for example, a central processing unit (CPU), a graphics processing unit (GPU), or a neural network processing unit (NPU). The hardware-implemented prediction apparatus 700 may include, for example, a microprocessor, a CPU, a processor core, a multi-core processor, a multiprocessor, an application-specific integrated circuit (ASIC), and a field-programmable gate array (FPGA).

The processor 730 may execute a program and control the prediction apparatus 700. A code of the program to be executed by the processor 730 may be stored in the memory 750.

Embodiments may be implemented using a hardware component, a software component, and/or a combination thereof. A processing device may be implemented using one or more general-purpose or special-purpose computers, such as, for example, a processor, a controller and an arithmetic logic unit (ALU), a digital signal processor (DSP), a microcomputer, an FPGA, a programmable logic unit (PLU), a microprocessor, or any other device capable of responding to and executing instructions in a defined manner. The processing device may run an operating system (OS) and one or more software applications that run on the OS. The processing device may also access, store, manipulate, process, and create data in response to execution of the software. For purpose of simplicity, the description of a processing device is singular; however, one of ordinary skill in the art will appreciate that a processing device may include multiple processing elements and multiple types of processing elements. For example, the processing device may include a plurality of processors, or a single processor and a single controller. In addition, different processing configurations are possible, such as parallel processors.

The software may include a computer program, a piece of code, an instruction, or some combination thereof, to independently or collectively instruct or configure the processing device to operate as desired. Software and/or data may be stored permanently or temporarily in any type of machine, component, physical or virtual equipment, or computer storage medium or device capable of providing instructions or data to or being interpreted by the processing device. The software may also be distributed over network-coupled computer systems so that the software is stored and executed in a distributed fashion. The software and data may be stored in a non-transitory computer-readable recording medium.

The methods according to the embodiments may be recorded in non-transitory computer-readable media including program instructions to implement various operations of the examples. The media may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of non-transitory computer-readable media include magnetic media such as hard disks, floppy disks, and magnetic tape; optical media such as CD-ROM discs and DVDs; magneto-optical media such as optical discs; and hardware devices that are specially configured to store and perform program instructions, such as read-only memory (ROM), random access memory (RAM), flash memory, and the like. Examples of program instructions include both machine code, such as produced by a compiler, and files containing higher level code that may be executed by the computer using an interpreter.

The above-described hardware devices may be configured to act as one or more software modules in order to perform the operations of the above-described embodiments, or vice versa.

Although the example embodiments have been described with reference to the limited drawings, one of ordinary skill in the art may apply various technical modifications and variations based thereon. For example, suitable results may be achieved if the described techniques are performed in a different order, and/or if components in a described system, architecture, device, or circuit are combined in a different manner, and/or replaced or supplemented by other components or their equivalents. Therefore, other implementations, other embodiments, and equivalents to the claims are also within the scope of the following claims and their equivalents.

While example embodiments have been described with reference to the figures, it will be understood by those of ordinary skill in the art that various changes in form and details may be made therein without departing from the spirit and scope as defined by the following claims and their equivalents.

What is claimed is:

1. A method of training a prediction model, the method comprising:
obtaining molecular descriptors of molecules based on a molecular database;
pre-training a pre-training neural network based on the molecular descriptors; and
adjusting the pre-training neural network such that the pre-training neural network matches a target task, by applying a training data set labeled corresponding to the target task to the pre-trained pre-training neural network.

2. The method of claim 1, wherein the pre-training of the pre-training neural network comprises:
reducing a dimensionality of the molecular descriptors based on a principal component analysis (PCA); and
pre-training the pre-training neural network based on the molecular descriptors with the reduced dimensionality as pseudo labels of a molecular graph.

3. The method of claim 2, wherein the reducing of the dimensionality of the molecular descriptors comprises generating a pre-training data set comprising molecular graphs respectively corresponding to the molecules and first latent vectors corresponding to the molecular graphs, by reducing the dimensionality of the molecular descriptors based on the PCA.

4. The method of claim 3, wherein the pre-training of the pre-training neural network comprises:
assigning the first latent vectors to pseudo labels of the molecular graphs respectively corresponding to the molecules; and
pre-training the pre-training neural network to predict a target pseudo label corresponding to the target molecule, based on the pseudo labels.

5. The method of claim 1, wherein the pre-training of the pre-training neural network comprises:
inputting input information corresponding to structural information of a target molecule to the pre-training neural network and outputting a molecular representation vector corresponding to the target molecule;
predicting a second latent vector corresponding to a target pseudo label of the input information by applying the molecular representation vector to a linear head; and
training at least one of the pre-training neural network or the linear head based on a difference between the first latent vectors and the second latent vector.

6. The method of claim 5, wherein the training of at least one of the pre-training neural network or the linear head comprises training at least one of the pre-training neural network or the linear head based on an objective function based on a weighted mean squared error (WMSE) between the first latent vectors and the second latent vector.

7. The method of claim 1, wherein the training data set labeled corresponding to the target task comprises a training data set labeled with a target chemical reaction corresponding to the target task and a target yield corresponding to the target chemical reaction.

8. The method of claim 1, wherein the pre-training neural network comprises at least one of a graph neural network (GNN) or a large language model (LLM).

9. The method of claim 1, wherein the target task comprises at least one of a prediction of a yield of a target chemical reaction corresponding to the target task, a prediction of a reaction condition of the target chemical reaction, or a prediction of physical properties of the target chemical reaction.

10. A method of predicting a target task, the method comprising:
receiving a query chemical reaction corresponding to a set of a reactant and a product; and
predicting a target task corresponding to the query chemical reaction by inputting the query chemical reaction to a prediction model comprising at least one pre-training neural network that is pre-trained,
wherein the prediction model is adjusted to predict a result corresponding to the target task by applying a training data set labeled corresponding to the target task to the pre-training neural network.

11. The method of claim 10, wherein the prediction model is configured to predict a yield corresponding to the query chemical reaction based on the query chemical reaction corresponding to the set of the reactant and the product being input.

12. The method of claim 11, wherein the reactant comprises molecular graphs corresponding to a plurality of reactant molecules corresponding to different reactions, and
wherein the product comprises a single molecular graph corresponding to a product molecule.

13. The method of claim 12, wherein the molecular graphs and the single molecular graph respectively comprise:
node vectors corresponding to node features corresponding to heavy atoms in a molecule; and
edge vectors corresponding to edge features corresponding to chemical bonds between the heavy atoms in the molecule.

14. The method of claim 13, wherein the node features comprise at least one of an atom type of the heavy atoms, formal charges of the heavy atoms, a degree of the heavy atoms, a hybridization of the heavy atoms, a number of atoms adjacent to the heavy atoms, a valence of the heavy atoms, a chirality of the heavy atoms, associated ring sizes of the heavy atoms, whether the heavy atoms donate or accept electrons, whether the heavy atoms are aromatic, or whether the heavy atoms include a ring.

15. The method of claim 13, wherein the edge features comprise at least one of a bond type of the chemical bonds between the heavy atoms, a stereochemistry of the chemical bonds between the heavy atoms, whether a ring is in the chemical bonds between the heavy atoms, or whether the chemical bonds between the heavy atoms are conjugated.

16. The method of claim 10, wherein the prediction model comprises:
the at least one pre-training neural network configured to output molecular query representation vectors by processing a query molecular graph within the query chemical reaction;
at least one fully-connected layer respectively corresponding to the at least one pre-training neural network and configured to output high-dimensional molecular representation vectors corresponding to the molecular query representation vectors; and
a feedforward neural network (FNN) configured to integrate the high-dimensional molecular representation vectors and output a prediction result corresponding to the target task by a representation vector of a chemical reaction obtained from the integrated high-dimensional molecular representation vectors.

17. The method of claim 16, wherein the prediction model further comprises a one-hot-encoding layer corresponding to each of a temperature condition, a pressure condition, and a solvent condition that correspond to the query chemical reaction, and
wherein the one-hot-encoding layer is between the at least one fully-connected layer and the FNN.

18. The method of claim 10, wherein the training data set labeled corresponding to the target task comprises a training data set labeled with a target chemical reaction corresponding to the target task and a target yield corresponding to the target chemical reaction.

19. A non-transitory computer-readable storage medium storing instructions that, when executed by a processor, cause the processor to perform the method of claim 1.

20. An apparatus for predicting a yield, the apparatus comprising:
a communication interface configured to receive a query chemical reaction corresponding to a set of a reactant and a product; and
a processor configured to predict a yield corresponding to the query chemical reaction by inputting the query chemical reaction to a prediction model comprising a pre-trained graph neural network (GNN),
wherein the prediction model is adjusted to predict the yield corresponding to the query chemical reaction by applying a training data set labeled corresponding to the predicted yield to the GNN.
